(19) United States
(12) Patent Application Publication
(10) Pub. No.: US 2005/0026992 A1
(43) Pub. Date: Feb. 3, 2005

(54) Treatment and prevention of cardiovascular events

(76) Inventors: Badal Kumar Sasmal, Hyderabad (IN); Billa Praveen Reddy, Hyderabad (IN); Vijay Dinanathji Nasare, Hyderabad (IN); Mailatur Sivaraman Mohan, Hyderabad (IN)

Correspondence Address: DR. REDDY'S LABORATORIES, INC., 200 Somerset Corporate Blvd, SEVENTH FLOOR,, BRIDGEWATER, NJ, 08807-2862, US

(21) Appl. No.: 10/898,549

(22) Filed: Jul. 28, 2004

(30) Foreign Application Priority Data
Jul. 28, 2003 (IN) 604/CHE/2003

(51) Int. Cl. A61K 31/401; A61K 31/366; A61K 31/225

(52) U.S. Cl. 514/423; 514/460; 514/548

(57) ABSTRACT

A pharmaceutical dosage form for treating or preventing cardiovascular events comprises therapeutic amounts of: a β-adrenergic receptor antagonist, a diuretic, or both; a cholesterol-lowering agent; an inhibitor of the renin-angiotensin system; and aspirin.

### INTRODUCTION TO THE INVENTION

The invention relates to a treatment for patients having an elevated risk of cardiovascular events and, more particularly, to a pharmaceutical composition for such treatment that combines a β-adrenergic receptor blocking agent with a cholesterol-lowering agent, an inhibitor of the renin-angiotensin system, and aspirin in a single dosage form, and to a method of preparing the pharmaceutical composition.

Cardiovascular diseases have been a leading cause of morbidity and mortality worldwide, being responsible for 16.6 million deaths in 2001. The majority (80 percent) of all deaths attributable to cardiovascular diseases (CVDs) are in low- and middle-income countries. By 2010, CVDs are expected to become the leading cause of mortality in developing countries. There is now a pressing need for developing and other countries to define and implement preventive interventions for CVDs.

A considerable fraction of the world population has been suffering serious life-threatening cardiovascular events, such as myocardial infarction (heart attack), cardiac arrest, congestive heart failure, stroke, peripheral vascular disease, and claudication. The risk factors associated with such life-threatening events include tobacco smoking, diabetes, elevated serum cholesterol, hypertension, systemic lupus erythematosus, prior heart attacks or strokes, hemodialysis, elevated homocysteine levels, obesity, sedentary lifestyles, receiving an organ transplant, and others.

The risk of having a cardiovascular event is not restricted to those with hypertension or hypercholesterolemia, but is continuous down to at least a blood pressure of 115/75 mm Hg and total cholesterol level of 4.0 mmol/l (about 155 mg/dL). However, it is only in the last few years that clinical trials have confirmed the benefits of blood pressure and cholesterol lowering in high-risk patients who do not have a clinical diagnosis of hypertension or hypercholesterolemia. Hence, the large majority of adults, and virtually all people with established vascular disease, would benefit from blood pressure and cholesterol lowering therapy, which would require simultaneous administration of blood pressure-reducing and cholesterol-lowering agents.

In a recent World Health Organization-Wellcome Trust meeting report, authors outlined the vast unmet needs in cardiovascular therapy and recommended the development of combination products for the same. The number of high-risk individuals who could benefit from affordable combination cardiovascular therapy in India and many other countries is substantial. In a combination product, each component causes a proportional risk reduction which is unaffected by the presence or absence of the other medicines. The long-term benefits would be even larger, perhaps more than a 75 percent overall risk reduction, since risk is only partially reversed in the first one to two years of blood pressure and cholesterol lowering treatment.

Considering the above unmet needs, it would be beneficial to have an effective and convenient therapy and formulations, comprising multiple cardiovascular disease-preventive medicines that would effectively reduce the risk of cardiovascular events. In conventional therapy, patients at higher risk of cardiovascular events frequently are on multiple drug therapy, taking two or more different medications at the same time. Presenting multiple medications in a single composition promotes patient compliance by avoiding the inconvenience of taking multiple doses of medicines in a single day, and reducing the chance of skipping doses.

U.S. Pat. No. 6,235,311 to Ullah et al. discloses a pharmaceutical composition which is useful for cholesterol lowering and reducing the risk of a myocardial infarction, which composition includes a statin, such as pravastatin, lovastatin, simvastatin, atorvastatin, cerivastatin or fluvastatin, in combination with aspirin, in a manner to minimize chemical interactions between aspirin and the statin, and to minimize the side effects of aspirin.

Weissman et al., in U.S. Pat. Nos. 6,121,249 and 6,323,188, disclose a method of reducing the incidence and severity of arteriosclerosis, atherosclerotic central nervous system disease, claudication, coronary artery disease, homocystine-related disorders, hypertension, peripheral vascular disease, presenile dementia, and restenosis in humans by daily administration of an effective amount of a combination of acetylsalicylic acid (ASA), at least one antioxidant, a cyanocobalamin compound (Vitamin B12), a folic acid compound, a pyridoxine compound (Vitamin B6), and a niacin compound.

U.S. Pat. No. 5,622,985 to Olukotun et al. discloses that inhibitors of 3-hydroxy-3-methylglutaryl coenzyme A (HMG CoA) reductase, also called “statins,” particularly pravastatin, when used alone or with an angiotensin converting enzyme (ACE) inhibitor, decrease the risk of a second heart attack in a patient who has a substantially normal cholesterol level.

U.S. Pat. No. 6,576,256 to Liang et al. discloses methods and compositions for reducing the risk of cardiovascular events in individuals who are at elevated cardiovascular risk, including individuals who have systemic lupus erythematosus. The methods comprise administering a combination of: a cholesterol-lowering agent, such as an HMG CoA reductase inhibitor; an inhibitor of the renin-angiotensin system, such as an ACE inhibitor; aspirin; and optionally one or more of vitamin B₆, vitamin B₁₂, and folic acid. Pharmaceutical formulations combining all the active agents in unit-dose form for once-daily dosing are also provided.

International Patent Publication WO 01/15674 of Aventis Pharma Deutschland GmbH relates to a combination of an inhibitor of the renin-angiotensin system, optionally an additional antihypertensive agent, a cholesterol-lowering agent, a diuretic, and aspirin, which can be administered to prevent cardiovascular events.

International Patent Publication WO 01/76632 by Wald and Law discloses a pharmaceutical formulation that contains at least two agents that lower blood pressure, having different modes of action, plus an active agent from at least two of: lipid regulating agents; platelet function altering agents; and serum homocysteine lowering agents. It is desired in this document to provide at least some of the drugs in smaller amounts than their customary therapeutic doses.

An article by N. J. Wald et al., “A Strategy to Reduce Cardiovascular Disease by More Than 80%,” British Medical Journal, Vol. 326, pp. 1419-1423, 2003, advocates the daily prophylactic treatment of everyone over age 55, and everyone with existing cardiovascular disease, with a “Polypill” containing the following six drugs: a drug to lower cholesterol, such as either atorvastatin (10 mg) or simvastatin (40 mg), the combination of three blood pressure lowering drugs from different classes, such as a thiazide, a β-blocker, and an ACE inhibitor (each at half the standard dose), folic acid (0.8 mg), and aspirin (75 mg).

Finally, in accordance with the recommendations made by the World Health Organization to develop combination products for cardiovascular therapy and test their efficacy in high risk individuals, it is highly desirable to develop a combination product using a variety of cardiovascular drugs including a β-adrenergic blocking agent, a diuretic, a cholesterol-lowering agent, an inhibitor of the renin-angiotensin system, aspirin, and optionally an anti-diabetes drug.

### SUMMARY OF THE INVENTION

For patients at elevated cardiovascular risk, convenient drug therapy was not available prior to this invention. In accordance with the said invention a combination of active agents of different categories is being provided, which can be conveniently administered once-daily to reduce a risk of cardiovascular event.

The present invention provides a once-daily stable oral dosage form containing a combination of a therapeutically effective dose of: a β-adrenergic receptor blocker, a diuretic, or both; a therapeutically effective dose cholesterol-lowering agent; a therapeutically effective dose of an inhibitor of the renin-angiotensin system; a therapeutically effective dose of aspirin; and optionally at least one of vitamin B₆, vitamin B₁₂, and folic acid; and a method for treating a patients at elevated cardiovascular risks by administering the dosage form on a daily basis.

In one aspect, the invention provides a pharmaceutical dosage form comprising therapeutic amounts of: a ,-adrenergic receptor antagonist, a diuretic, or both; a cholesterol-lowering agent; an inhibitor of the renin-angiotensin system; and aspirin.

In another aspect, the invention provides a pharmaceutical dosage form comprising therapeutic amounts of: a β-adrenergic receptor antagonist, a diuretic, or both; a cholesterol-lowering agent; an inhibitor of the renin-angiotensin system; and aspirin; wherein acidic components are separated from basic components.

In a further aspect, the invention provides a tablet comprising two layers, wherein a first layer comprises simvastatin and aspirin, and a second layer comprises atenolol and lisinopril.

In a still further aspect, the invention provides a tablet comprising two layers, wherein the first layer comprises simvastatin and aspirin, and a second layer comprises hydrochlorothiazide and lisinopril.

### DETAILED DESCRIPTION OF THE INVENTION

In accordance with the present invention, a pharmaceutical composition is provided which includes a β-adrenergic receptor blocker, a cholesterol-lowering agent, an inhibitor of the renin-angiotensin system, and aspirin, with or without a diuretic, which reduces risk of cardiovascular event with minimal or no physical and chemical incompatibility, and gives reduced side effects normally associated with use of such drugs.

When the terms “active agent,” “pharmacologically active agent,” and “drug” are used, it is to be understood that this includes the active molecule as well as pharmaceutically acceptable, pharmacologically active, salts, esters, amides, prodrugs, metabolites, analogs, etc. Many drug substances are formulated using such other forms, but only the active portion will be identified herein.

The term “therapeutic amount” in connection with a drug indicates the amount of the drug contained in a daily dose, as customarily prescribed for a primary indication that is within the scope of this invention. These amounts are conveniently summarized for many drugs in the “BNF Recommended Dose” column of tables on pages 11-17 of WO 01/76632 (the data in the tables being attributed to the March 2000 British National Formulary) and can also be found in other standard formularies and other drug prescribing directories. For some drugs, the customary prescribed dose for an indication will vary somewhat from country to country.

β-adrenergic receptor antagonists block the action of the sympathetic nervous system and a portion of the involuntary nervous system. By blocking the action of these nerves, they reduce the heart rate and are useful in treating abnormally rapid heart rhythms. These drugs also reduce the force of heart muscle contractions and lower blood pressure. By reducing the heart rate and the force of muscle contraction, β-blockers reduce heart muscle oxygen demand.

Useful β-adrenergic blocking agents are selected from a group including atenolol, betaxolol, acebutolol, bisoprolol, carteolol, labetalol, metoprolol, nadolol, oxprenolol, penbutolol,pindolol, propranolol, sotalol, and timolol. Atenolol is a presently preferred β-adrenergic blocking agent.

This invention employs any effective cholesterol-lowering agent or combination of such agents. Useful cholesterol-lowering agents include HMG CoA reductase inhibitors, bile acid sequestrants, probucol, and fibric acid agents. Also useful is the selective inhibitor of intestinal cholesterol absorption having the adopted name “ezetimibe,” and the chemical name 1-(4-fluorophenyl)-3(R)-[3-(4-fluorophenyl)-3(S)-hydroxypropyl]-4(S)-(4-hydroxyphenyl)-2-azetidinone. Ezetimibe is particularly effective when administered together with a statin.

Preferred are the HMG CoA reductase inhibitors. These agents are competitive inhibitors of HMG CoA reductase, the rate-limiting step in cholesterol biosynthesis. They occupy a portion of the binding site of HMG CoA, blocking access of this substrate to the active site on the enzyme. HMG CoA reductase inhibitors comprise atorvastatin, cerivistatin, fluindostatin, fluvastatin, lovastatin, mevastatin, pravastatin, simvastatin, and velostatin; the most preferred agents are lovastatin and pravastatin, particularly lovastatin.

The renin-angiotensin system plays a major role in regulating blood pressure. Renin, an enzyme, functions by acting on angiotensinogen to form the decapeptide angiotensin I. Angiotensin I is rapidly converted to the octapeptide angiotensin II by angiotensin converting enzyme (ACE). Angiotensin II acts by numerous mechanisms to raise blood pressure, including raising total peripheral resistance. Inhibitors of the renin-angiotensin system are classified as angiotensin converting enzyme (ACE) inhibitors and angiotensin II receptor antagonists (ARBs).

Examples of angiotensin converting enzyme (ACE) inhibitors are captopril, cilazapril, delapril, enalapril, fentiapril, fosinopril, indolapril, lisinopril, perindopril, pivopril, quinapril, ramipril, spirapril, trandolapril, and zofenopril; preferred for use in this invention are captopril, enalapril, fosinopril, lisinopril, quinapril, ramipril, and trandolapril, and more preferred is enalapril. Useful angiotensin II receptor antagonists include losartan, irbesartan, eprosartan, candesartan, valsartan, telmisartan, zolasartin, and tasosartan. Preferred is losartan. In this invention, angiotensin converting enzyme (ACE) inhibitors are more preferred over angiotensin II receptor antagonists.

Cyclooxygenase inhibitors are useful in the present invention, due to their ability to affect platelets; the most widely used and studied cyclooxygenase inhibitor is aspirin, which has been shown to prevent myocardial infarction and strokes due to thrombosis, when administered in low daily doses over a long term to patients at risk for cardiovascular events. When sufficient aspirin is present in the circulatory system, platelets that are being formed have an impaired ability to aggregate over their entire 7-10 day lifetimes.

Diuretics increase the rate of urine flow and sodium excretion and are used to adjust the volume and/or composition of body fluids in a variety of clinical situations, including hypertension, congestive heart failure, renal failure, nephritic syndrome and cirrhosis. Diuretics can be selected from variety of classes such as inhibitors of carbonic anhydrase, loop diuretics, thiazides and thiazide-like diuretics, K⁺ sparing diuretics, and antagonists of mineralocorticoid receptors.

In an embodiment of this invention thiazides and thiazide-like derivatives are preferred diuretics, including bendroflumethazide, chlorothiazide, hydrochlorothiazide, hydroflumethazide, methyclothazide, polythiazide, trichlormethazide, chlorthalidone, indapamide, metolazone, and qiunethazone. Presently, the most preferred diuretic is hydrochlorothiazide, which acts by blocking salt and fluid reabsorption in the kidneys, causing increased urine output (diuresis). It has also been widely used in treating mild hypertension.

Further, a combination product can include at least one antidiabetic agent, such as the oral hypoglycemic agents metformin, the sulfonylurea drugs glibenclamide, tolbutamide, tolazamide, glyburide, glipizide, and glimipiride, and the thiazolidinedione drugs troglitazone, rosiglitazone, and pioglitazone. These generally act to improve insulin utilization by the cells, and (in some instances) stimulate insulin production by the pancreas or decrease hepatic glucose production. An antidiabetic agent can be included in a product that is intended for use by persons having non-insulin dependent diabetes mellitus.

Elevated serum levels of homocysteine are highly correlated with atherosclerosis, heart disease, stroke, and peripheral vascular disease. Vitamin B₆, vitamin B₁₂, and folic acid act to lower homocysteine levels and reduce the incidence of these disease states. Vitamin B₆ is included in amounts between about 2 mg and 2 grams. Vitamin B₁₂ will be included in amounts between about 3 μg and 2 mg. Folic acid will generally be included in amounts up to about 5 mg, such as about 400 to 800 μg, about 500 μg to 2 mg, or about 1 mg to 5 mg.

It should be recognized that the foregoing lists of drugs in their particular classes are not exhaustive, and that other drugs will also be useful in the invention. In general, it is desired to use drugs that can be dosed once-daily, either due to their pharmacokinetic characteristics or due to their ability to be formulated in controlled release forms, to facilitate patient compliance with the dosing regimen.

In accordance with an embodiment of the invention, various dosage forms that can effectively administer the drug combination include tablets, capsules, and caplets, and may also comprise a plurality of granules, beads, powders, or pellets that may or may not be encapsulated.

Various pharmaceutical compositions and processes of preparation are set forth in a description that follows, where the terms “a said combination” or “a combination” indicates combinations comprising therapeutically effective unit dosages of β-adrenergic receptor blocking agent, cholesterol-lowering agent,. inhibitor of the renin-angiotensin system, aspirin, and optionally one or more vitamins like vitamin B₆, vitamin B₁₂, folic acid or a combination thereof, a diuretic, and/or a hypoglycemic drug.

Combining two or more active ingredients in single dosage form has critical considerations, due to the possibility of chemical interactions between the drug substances. Acidic active ingredients like aspirin can react with basic drugs, and acidic ingredients such as aspirin can facilitate the degradation of acid labile drugs including lovastatin and pravastatin. In the invention, such drug interactions were considered and interacting active ingredients were physically separated using various approaches given below.

1. Multiple layer tablet or press-coated tablets. In a combination where drugs such as aspirin and enalapril maleate are the acidic drugs, and drugs such as atenolol and lovastatin are the basic drugs, the acidic and basic substances can be physically separated as two distinct or isolated layers in a compressed tablet, or in the core and shell of a press-coated tablet. Hydrochlorthiazide, being compatible with acidic as well as basic drugs, has the flexibility of being placed in either layer.

In a further embodiment of a multiple layer composition at least one active ingredient can be enteric-coated. In a still further embodiment of a multiple layer composition, at least one active ingredient can be presented in a controlled release form.

Another useful arrangement is to provide a combination in three or more physically isolated segments of a compressed tablet. The multiple layer tablet may be film coated.

2. Tablets or Capsules comprising a plurality of beads, granules, or pellets. All active ingredients including the vitamins of the combination are formulated into granules or beads or pellets that are further coated with a protective coat, an enteric coat, or a film coat to avoid the possible chemical interactions. Granulation and coating of granules or beads is done using techniques well known to a person skilled in the art. At least one active ingredient can present in a controlled release form. Finally these coated granules or beads are filled into hard gelatin capsules or compressed to form tablets.

3. Capsules comprising microtablets or minitablets of all active ingredients. Microtablets were prepared of individual components of a said combination were prepared using well known pharmaceutical procedures of tablet making like direct compression, dry granulation or wet granulation. All these individual microtablets were filled into hard gelatin capsules. A final dosage form may comprise one or more than one microtablet of each individual component. Further, these microtablets may be film coated or enteric coated.

4. Capsule comprising one or more microtablets and powder, or one or more microtablets and granules or beads. In order to avoid interactions between drugs, some active ingredients of a said combination can be formulated as microtablets and the others filled into capsules as a powder, granules, or beads. A microtablet can be film coated or enteric coated. At least one active constituent can be presented in sustained release form. 5. Active ingredient distributed in inner and outer phase in tablets. In an attempt to divide chemically incompatible components of proposed combination, few interacting components are converted in granules or beads using well known pharmaceutical procedures in prior art. The prepared granules or beads (inner phase) are then mixed with outer phase comprising the remaining active ingredients and at least one pharmaceutically acceptable excipients. The mixture thus comprising inner and outer phase is compressed into tablets or molded into tablets.

In further embodiments included within this approach, the granules or beads can be controlled release or immediate release.beads or granules, and can further be coated using an enteric polymer in an aqueous or non-aqueous system, using methods and materials that are known in the art, if required. 6. Single dosage unit comprising suitable buffering agent. All powdered ingredients of said combination are mixed and a suitable quantity of one or more buffering agents is added to the blend to minimize possible interactions.

In the above described approaches, final coated dosage forms, particularly tablets, are film coated with 1-8%, more preferably 2-6% by weight of a polymer such as a cellulose ether, an acrylic such as a methacrylate and methyl methylacryate copolymer, or a vinyl such as polyvinyl alcohol. Enteric coatings mentioned above usually provide a 5-15% weight buildup, over the uncoated tablets or pellets, of enteric coating polymers such as shellac, a polymethacrylate, hydroxypropyl methylcellulose phthalate, polyvinyl acetate phthalate, and cellulose acetate phthalate. Many other coating substances, and the techniques that are suitable for applying coatings to particles, are known in the art and can be used in the practice of this invention.

The following examples are representative embodiments of the invention, and are not to be construed as limiting the scope of the invention as defined by the appended claims, but serve only to show how various approaches to develop a combination product are practically carried out.

### EXAMPLE 1

Bi-layer tablets weighing 360 mg were prepared using the following:

| Ingredients | mg/Tablet |
|---|---|
| First layer: | |
| Enteric coated aspirin (granular) | 75 |
| Enalapril maleate | 5 |
| Lactose | 64.5 |
| Microcrystalline cellulose | 21.5 |
| Croscarmellose sodium | 8 |
| Colloidal silicon dioxide | 3 |
| Zinc stearate | 3 |
| Second layer: | |
| Atenolol | 25 |
| Lovastatin | 20 |
| Butylated hydroxyanisole | 1.75 |
| Hydrochlorothiazide | 6.25 |
| Lactose | 46 |
| Microcrystalline cellulose | 50 |
| Red iron oxide | 1 |
| Povidone K90 | 5 |
| Croscarmellose sodium | 6 |
| Zinc stearate | 4 |
| Colloidal silicon dioxide | 5 |
| Film coat: | |
| Coating material | 10 |

To prepare the tablets, the first layer components were combined and blended to achieve uniformity. Separately, the second layer components atenolol, lovastatin, hydrochlorothiazide, lactose, microcrystalline cellulose (as AVICEL™ PH 101 from FMC Corporation, Philadelphia, Pa. U.S.A.), and iron oxide were sifted through a sieve and mixed to uniformity, then an aqueous isopropanol solution containing povidone and buylated hydroxyanisole was used to granulate the powder mixture, which was then dried and crushed and croscarmellose sodium, silicon dioxide, and zinc stearate were added and blended.

The first layer mixture and the second layer mixture were sequentially compressed in a die to form bi-layer tablets, which finally were film coated.

### EXAMPLE 2

A hard gelatin capsule containing a microtablet, a minitablet, and powder was prepared using the following:

| Ingredients | mg/Dosage Form |
|---|---|
| Microtablet | |
| Enalapril Maleate | 5 |
| Lactose anhydrous | 40 |
| Microcrystalline Cellulose | 9 |
| Maleic acid | 0.5 |
| Zinc stearate | 0.5 |
| Hydroxypropyl methylcellulose | 1.1 |
| Minitablet | |
| Enteric coated aspirin (granular) | 75 |
| Croscarmellose sodium | 5 |
| Stearic acid | 1 |
| Enteric coat | 3.2 |
| Powder | |
| Atenolol | 25 |
| Lovastatin | 20 |
| Hydrochlorothiazide | 6.25 |
| Microcrystalline cellulose | 25 |
| Dibasic calcium phosphate, anhydrous | 25 |
| Calcium carbonate | 10 |
| Talc | 2 |
| Magnesium stearate | 1.5 |
| Colloidal silicon dioxide | 1.5 |

A film coated microtablet of enalepril maleate was prepared by blending the first five listed ingredients, compressing to form a tablet, coating with a solution of hydroxypropyl methylcellulose, and drying. An enteric-coated minitablet of aspirin was prepared by mixing the first three listed ingredients, compressing to form a tablet, coating with an enteric polymer solution, and drying. A powder was prepared by blending atenolol, lovastatin, hydrochlorthiazide and the listed excipients. Then the microtablet, the minitablet and the powder were filled into a size 0 hard gelatin capsule.

### EXAMPLE 3

Capsules containing a combination of cardiovascular drugs were prepared using the following:

| Ingredients | mg/Capsule |
|---|---|
| Enalapril maleate | 5 |
| Enteric coated aspirin (granular) | 75 |
| Atenolol | 25 |
| Lovastatin | 20 |
| Hydrochlorothiazide | 6.25 |
| Folic acid | 5 |
| Lactose anhydrous | 30 |
| Calcium carbonate | 30 |
| Magnesium oxide | 25 |
| Magnesium stearate | 2 |
| Colloidal silicon dioxide | 1 |

Lovastatin, atenolol, enalapril maleate, hydrochlorthiazide, aspirin, folic acid and lactose were mixed uniformly, and to this mixture calcium carbonate and magnesium oxide were added followed by further mixing. Magnesium stearate and silicon dioxide were added to the dry mixture and blended to uniformity, and the final powder was filled into a hard gelatin capsule.

### EXAMPLE 4

Tablets containing a combination of drugs were prepared using the following:

| Ingredient | mg/Tablet |
|---|---|
| First layer | |
| Simavastatin | 20 |
| Lactose monohydrate | 157 |
| Butylated hydroxyanisole | 0.08 |
| Ascorbic acid | 10 |
| Citric acid anhydrate | 5 |
| Pregelatinized starch | 20 |
| Microcrystalline cellulose | 20 |
| Zinc stearate | 2.5 |
| Aspirin | 75 |
| Second layer | |
| Atenolol | 50 |
| Lisinopril | 10 |
| Dibasic calcium phosphate | 89.94 |
| Microcrystalline cellulose | 40 |
| Mannitol | 25 |
| Ponceau 4R red dye | 0.5 |
| Povidone | 4 |
| Zinc stearate | 1 |
| Sodium starch glycolate | 8 |
| Film coating | |
| Coating material | 10 |

To prepare the tablets, the first seven ingredients were blended, then were moistened and granulated, dried, and the next two ingredients were added and blended to form a first layer mixture. A second layer mixture was prepared by dry granulating the first five second layer ingredients, the povidone, and half of the dye and zinc stearate; after granules were formed, the remainder of the dye and zinc stearate, plus all of the sodium starch glycolate, were added and blended. Finally, the first layer mixture was compressed in a die, then the second layer mixture was added and compressed to form a two-layer tablet, then the tablet was film coated and dried.

Alternatively, the second layer mixture has been prepared by wet granulating the first five second layer ingredients, plus povidone; after drying and milling, all of the zinc stearate, dye and sodium starch glycolate were added and blended.

### EXAMPLE 5

Tablets containing a combination of drugs were prepared using the following:

| Name of ingredient | mg/Tablet |
|---|---|
| First layer | |
| Simavastatin | 40 |
| Lactose monohydrate | 157 |
| Butylated hydroxyanisole | 0.08 |
| Ascorbic acid | 10 |
| Citric acid anhydrate | 5 |
| Pregelatinized starch | 20 |
| Microcrystalline cellulose | 20 |
| Zinc stearate | 2.5 |
| Aspirin | 75 |
| Second layer | |
| Hydrochlorothiazide | 12.5 |
| Lisinopril | 10 |
| Dibasic calcium phosphate | 89.94 |
| Microcrystalline cellulose | 40 |
| Mannitol | 25 |
| Povidone | 4 |
| Ponceau 4R red dye | 1 |
| Sodium starch glycolate | 8 |
| Zinc stearate | 1 |
| Film coating | |
| Coating material | 10 |

To prepare the tablets, the first layer ingredients were prepared as in preceding Example 4. The first six second layer ingredients, plus half of the zinc stearate and dye, were subjected to dry granulation, then the other half of the zinc stearate and dye, plus all of the sodium starch glycolate, were added and blended. Tableting was done as in Example 5, followed by film coating and drying.

As in Example 5, the first six second layer ingredients alternatively can be wet granulated, dried, and milled, and then the final three ingredients added and blended.

1. A pharmaceutical dosage form comprising therapeutic amounts of: a β-adrenergic receptor antagonist, a diuretic, or both; a cholesterol-lowering agent; an inhibitor of the renin-angiotensin system; and aspirin.

2. The pharmaceutical dosage form of claim 1, comprising a β-adrenergic receptor antagonist.

3. The pharmaceutical dosage form of claim 1, comprising a diuretic.

4. The pharmaceutical dosage form of claim 1, wherein the β-adrenergic receptor antagonist comprises atenolol.

5. The pharmaceutical dosage form of claim 1, wherein the cholesterol-lowering agent comprises an inhibitor of HMG CoA reductase.

6. The pharmaceutical dosage form of claim 1, wherein the cholesterol-lowering agent comprises lovastatin.

7. The pharmaceutical dosage form of claim 1, wherein the cholesterol-lowering agent comprises simvastatin.

8. The pharmaceutical dosage form of claim 1, wherein the inhibitor of the renin-angiotensin system comprises an ACE inhibitor.

9. The pharmaceutical dosage form of claim 1, wherein the inhibitor of the renin-angiotensin system comprises enalapril.

10. The pharmaceutical dosage form of claim 1, wherein the inhibitor of the renin-angiotensin system comprises lisinopril.

11. The pharmaceutical dosage form of claim 1, further comprising an agent to lower homocysteine levels.

12. The pharmaceutical dosage form of claim 1, further comprising folic acid.

13. The pharmaceutical dosage form of claim 1, further comprising a hypoglycemic agent.

14. The pharmaceutical dosage form of claim 1, comprising atenolol, lovastatin, enalapril, aspirin, and hydrochlorothiazide.

15. A pharmaceutical dosage form comprising therapeutic amounts of: a β-adrenergic receptor antagonist, a diuretic, or both; a cholesterol-lowering agent; an inhibitor of the renin-angiotensin system; and aspirin; wherein acidic components are separated from basic components.

16. The pharmaceutical dosage form of claim 15, further comprising a diuretic.

17. The pharmaceutical dosage form of claim 15, wherein acidic components are present in one layer of a multilayer tablet, and basic components are present in another layer.

18. The pharmaceutical dosage form of claim 15, wherein acidic components are present in one of a core and a shell of a multiple-compressed tablet, and basic components are present in the other of a core and a shell.

19. The pharmaceutical dosage form of claim 15, comprising an acidic component mixture formed into a particle.

20. The pharmaceutical dosage form of claim 15, comprising a basic component mixture formed into a particle.

21. The pharmaceutical dosage form of claim 15, comprising an acidic component mixture formed into a particle and a basic component mixture formed into another particle.

22. The pharmaceutical dosage form of claim 15, wherein at least one of an acidic component mixture and a basic component mixture is formed into a particle and coated with a polymer.

23. A tablet comprising two layers, wherein a first layer comprises simvastatin and aspirin, and a second layer comprises atenolol and lisinopril.

24. A tablet comprising two layers, wherein the first layer comprises simvastatin and aspirin, and a second layer comprises hydrochlorothiazide and lisinopril.
